(19) United States
(12) Patent Application Publication
(10) Pub. No.: US 2025/0318791 A1
(43) Pub. Date: Oct. 16, 2025

(54) TOPOGRAM-BASED LOCALIZATION AND/OR ATTENUATION MAPPING IN EMISSION TOMOGRAPHY

(71) Applicant: Siemens Medical Solutions USA, Inc., Malvern, PA (US)

(72) Inventors: Francesc dAssis Massanes Basi, Chicago, IL (US); Alexander Hans Vija, Evanston, IL (US); Wendy McDougald, Schaumburg, IL (US)

(21) Appl. No.: 18/632,353

(22) Filed: Apr. 11, 2024

(51) Int. Cl. A61B 6/03; G06T 7/00

(52) U.S. Cl. CPC A61B 6/037; G06T 7/0012; G06T 2207/10108; G06T 2207/30048

(57) ABSTRACT

In emission imaging, one, two, or a few topograms without a full CT scan are used to localize, such as through triangulation, and/or generate an attenuation map, such as based on model fitting. A limited radiation exposure, rather than a longer, larger full CT exposure, is needed to localize and/or provide attenuation correction for emission imaging, such as cardiac SPECT imaging.

### BACKGROUND

The present embodiments relate to emission tomography. In emission tomography, an added radioisotope in a patient gathers at locations of metabolic function. The added radioisotope causes emissions. The emission tomography system detects the emissions. Various approaches for detecting emissions may be used, such as Single Photon Computed Tomography (SPECT) or Positron Emission Tomography (PET).

In some clinical specific tasks, there is the need to perform acquisition planning, such as centering of a focusing collimator in the organ area. Where a simple depth and localization is needed for SPECT imaging in general, the emission data may be used to localize anatomy. For cardiac SPECT imaging, a focusing collimator may not generate detectable contrast above background to determine the anatomy. FIG. 1 shows an example imaging monitor scan (counts of emissions from a patient). Anatomy is difficult to determine in some situations, such as where the contrast in early onset diseases with radiopharmaceutical (e.g., PYP) assessment is close to 1. To get the anatomical information needed, the CT in the SPECT/CT system is used.

The emissions pass through the body of the patient, so are subject to attenuation. For a ‘sharper’ spatial resolution of anatomical information in experimental arrangements, higher energy and longer duration computed tomography (CT) acquisition is used for calculating attenuation corrections. Generally, CT imaging uses a low dose topogram/scout for scan positioning and then a higher dose acquisition using a helical or spiral scan for calculation of attenuation corrections. CT imaging applies radiation to the patient. To reconstruct SPECT cardiac data accurately and obtain reliable quantification information, (Bq/ml), a CT attenuation correction map is required. A clinical cardiac/chest CT acquisitions will expose patients upwards to an approximate effective radiation dose of 8.7 mSv.

Dose reduction may be achieved by system side modification and CT tube current and spectral changes (i.e., Energy (kVp), Flux (mAs), beam shape, and filter (bow tie, Sn, etc. modification)) or by image reconstruction techniques, such as compressed sensing, filtering, and other noise reduction and resolution stabilizing approaches. However, undesired radiation dose is still delivered. Model-based and emission tomography-based efforts to estimate the attenuation map are directed to specific clinical tasks given available data, so may not be applicable for cardiac and other emission imaging.

### SUMMARY

By way of introduction, the preferred embodiments described below include methods, systems, instructions, and computer readable storage media for localization and/or attenuation map generation in emission imaging, such as SPECT imaging. One, two, or a few topograms without a full CT scan are used to localize, such as through triangulation, and/or generate an attenuation map, such as based on model fitting. A limited radiation exposure is needed to localize and/or provide attenuation correction for emission imaging, such as cardiac SPECT imaging.

In a first aspect, a method is provided for localization for cardiac imaging in a SPECT system. First and second topograms of a patient are acquired. The first and second topograms are x-ray images captured from different angles relative to a patient. The first and second topograms are acquired without a full computed tomography acquisition. A cardiac location of interest is determined in three dimensions from the first and second topograms. A collimator of the SPECT system is focused based on the cardiac location determined from the first and second topograms. Using the collimator as focused, the SPECT system images the cardiac location.

In a second aspect, a system is provided for localization and/or attenuation mapping for emission imaging. An x-ray-based scanner is configured to acquire one or more x-ray topograms from respective one or more angles to a patient separate from any helical or spiral computed tomography scan. An emission scanner is configured to detect emissions from the patient. A processor is configured to localize anatomy relative to the emission scanner and/or generate an attenuation map from the one or more topograms and cause the emission scanner to detect the emissions based on the localization and/or attenuation map.

In a third aspect, a method is provided for attenuation mapping in a SPECT scanner. First and second topograms of a patient are acquired. The first and second topograms are x-ray images captured from different angles relative to a patient over a scan of less than three seconds. An attenuation map is generated from the first and second topograms. The SPECT scanner images based on the attenuation map.

The illustrative embodiments below summarize other aspects or features of the first, second and third aspects above. Aspects or features used for one type of claim (e.g., method or system) may be used in other types.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is an example patient imaging monitor image from a SPECT scanner;

FIG. 2 is a flow chart diagram of one embodiment of a method for localization and/or attenuation mapping in an emission tomography system;

FIG. 3 illustrates various features of the method of FIG. 2;

FIG. 4 illustrates cardiac SPECT imaging with localization using user selection and localization based on topogram triangulation; and

FIG. 5 is a block diagram of one embodiment of a emission tomography imaging system with localization and/or attenuation map generation.

### DETAILED DESCRIPTION OF THE DRAWINGS AND PRESENTLY PREFERRED EMBODIMENTS

Localization and/or attenuation map generation is provided based on a limited number of topograms in emission tomography imaging, such as SPECT. For example, SPECT-based cardiac quantification relies on topogram generated attenuation. The tasks of localization and/or attenuation map generation may be constrained to using the diagnostic CT or another x-ray imager on the respective SPECT/CT device. One, two, or more topograms are acquired, but less x-ray scanning is used than typical combined lower dose maximum non-colinear topograms for CT imaging. Rather than a full CT scan, such as the tens or hundreds of images acquired in a helical or spiral CT scan, the limited number of topograms results in less dose. The CT scout, topogram, or other positioning scan with the tube at a fixed location (e.g., 3, 9, or 12 o'clock) is used rather than the following full CT scan. Relying on internal mechanical referencing between the CT and SPECT scanners, the topograms may be used for the estimation of cross modality localization of a point for SPECT scanning. The topograms may, additionally or instead, be used to generate an attenuation map.

In one implementation, two topograms are acquired at a much lower radiation dose than the typical combined non-colinear topograms for CT. CT data information is acquired at low X-ray exposures, such as the two topograms exposing the patient to 1 mSv or less. The topograms may be acquired in a few seconds (3 or less) or less than one minute, which is much more rapidly than the 3 or more minutes for a CT scan. The topogram has a longer axial extent (e.g., 512 mm or 1024 mm) than a CT scan. The topogram or positioning scan is used to define the range of the CT. A CT typically only cover the body portion of interest, in this case the heart, with axial padding (e.g., around 250 mm). The topograms are used for localization and/or attenuation coefficient mapping at a lower dose (x-ray exposure) to the patient and/or scan time than a full CT. Using two or a limited number of topograms reduces overall patient absorbed radiation doses from CT cardiac imaging.

The information extracted from the topogram data is used for localization. Anatomy is pinpointed for subsequent use in emission imaging. The topograms provide anatomical referencing based on triangulation or other spatial identification of anatomy of interest. Additionally, or alternatively, the information extracted from the topogram data is used for estimation of an attenuation map for the purpose of localization, rigid 3D registration (e.g., external modality image, e.g., CT or magnetic resonance), and/or attenuation correction for emission tomography reconstruction.

In the example use case of cardiac centering, the topograms are used to center a collimator (e.g., non-parallel hole or focusing collimator) to focus on the heart of the patient. Using the topograms for localization reduces X-ray dose to the patient and improves the use of the focusing collimator through more accurate localization than provided with emission imaging monitor images. For example, early stages of cardiac amyloid disease may have little radiotracer uptake compared to the background (see FIG. 1) so show little anatomical information for localization. By using topograms, the heart is better localized for later SPECT imaging. The CT scanner is used to acquire a few topograms for focusing the collimator on the anatomy of interest. The SPECT detector is aligned more accurately than anatomical guesses based on the imaging monitor images. For example, two low dose topograms (e.g., top and lateral) are acquired. The coordinates of anatomy of interest in a coordinate system of the CT scanner are determined from the topograms (e.g., by triangulation) and recorded. The CT coordinates are translated to the emission tomography coordinate system. The collimator and detector of the emission scanner are accurately positioned to image the heart or part of the heart based on the localization.

The cardiac SPECT imaging example is used herein. The localization and/or attenuation map generation may be applied in other SPECT imaging contexts (e.g., for imaging other anatomy such as myocardial blood flow to determine flow reserve). Where an injection is used, the SPECT scan is to start as soon as the injection is triggered with a small bolus delay where the activity has not yet reached the heart (is not yet visible in the heart). In dynamic imaging, the SPECT is ready and at the desired cardiac start position to acquire when the bolus enters the heart. Non-dynamic imaging may be used. The topograms provide a 3D location of organs and/or may be used to generate an attenuation map whilst allowing for the reduction of patient absorbed X-ray doses as compared to a full CT scan. Additionally, this imaging data set (i.e., topograms) may be applied to patients requiring multiple diagnostic, prognostic, or treatment planning imaging acquisitions to potentially eliminate additional CT scans.

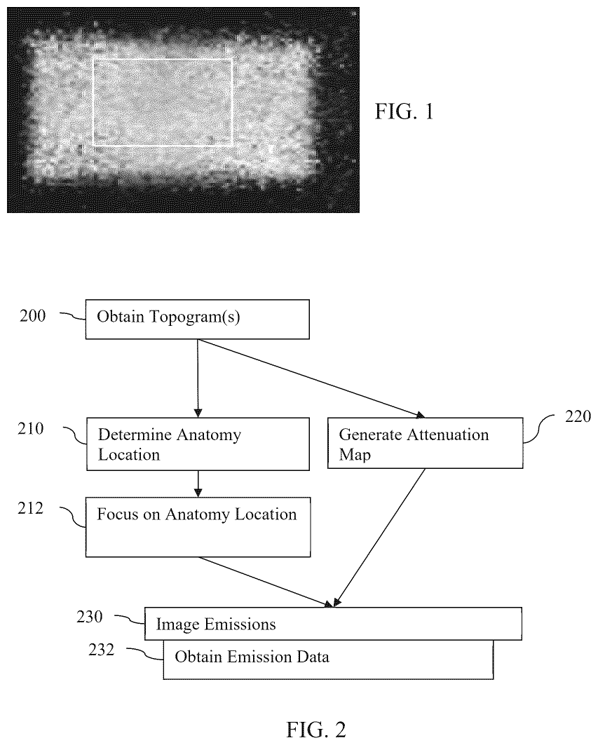

FIG. 2 shows one embodiment a method for localization and/or attenuation mapping in an emission tomography system, such as a SPECT system. Topograms are used for localization instead of a full CT scan or emission data. Alternatively, or additionally, topograms are used for attenuation mapping instead of a full CT scan. Topograms provide sufficient information for localization and/or attenuation mapping while limiting radiation dosage.

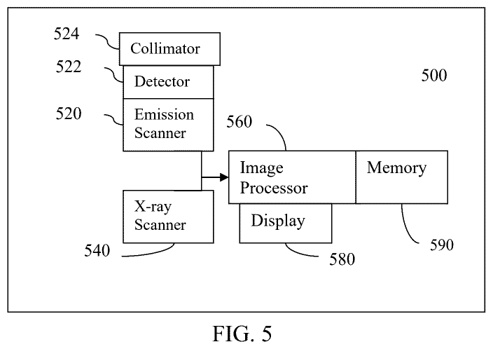

The method of FIG. 2 is implemented using the system of FIG. 5, an image processor, a computer, a CT scanner, x-ray scanner, a functional MR scanner, a SPECT imager, a PET imager, a server, and/or another device. For example, a CT scanner performs act 200, and a SPECT scanner performs acts 230 and 232. A computer, server, or another image processor, whether part of the CT or emission tomography scanner or not, performs acts 210, 212, and/or 220. Other devices may be used, such as display for displaying images or a user input for receiving designation of a location of anatomy of interest in topograms.

The method is performed in the order shown (numerical or top to bottom), but other orders may be used. For example, acts 210 and 212 are performed simultaneously or in any sequence with act 220. As another example, act 232 is performed prior to or as part of act 230, which may be performed, at least in part, prior to act 220.

Additional, different, or fewer acts may be provided. For example, acts for configuring the CT scanner and/or emission tomography scanner are included. As another example, act 220 is provided without acts 210 and 212, or acts 210 and 212 are provided without act 220. In yet another example, an act for calibrating the CT scanner to the emission tomography scanner is provided.

In act 200, an x-ray imager, such as an x-ray scanner or CT scanner, obtains one, two, or more topograms of a patient. X-rays are transmitted, and the x-rays passing through the patient are detected. The topograms may be obtained from memory or transfer over a computer network in other implementations.

The topograms are x-ray images. X-ray images represent attenuation along x-ray lines from the source to the detector so represent the patient in two dimensions based on tissue in three dimensions. The x-ray images are projections along the view direction into a two-dimensional (2D) image. The topogram or topogram data represents anatomical information about the examined patient (e.g., shape, volume, thickness, and/or density of tissue types) in 2D.

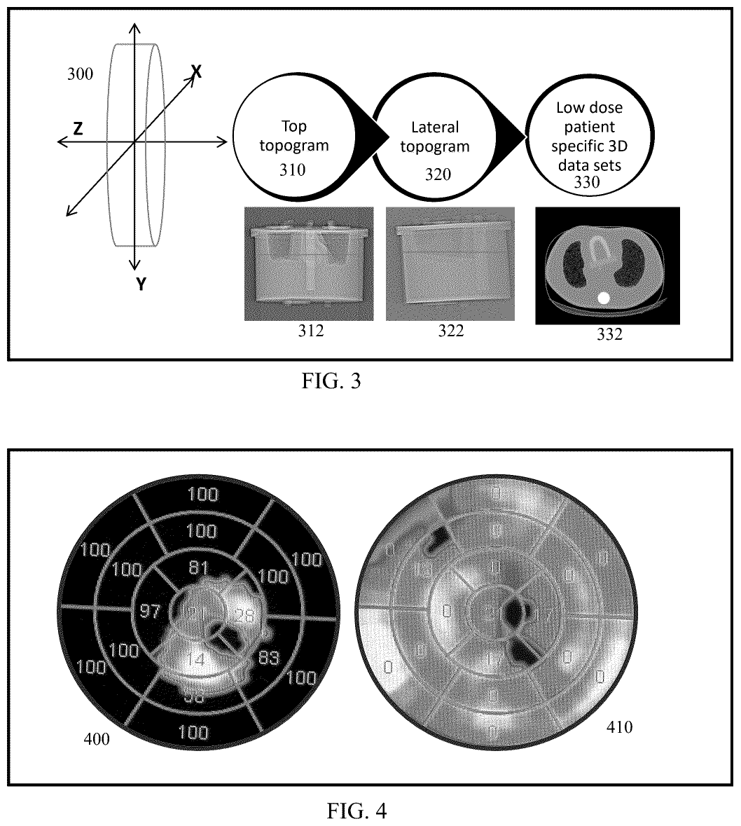

These topograms have a field of view of most of the patient or torso of the patient. The topograms are taken with the source and detector at particular orientations relative to the patient. The normal vector to the detector is a normal to the x-ray image. Different topograms are acquired from different angles relative to the patient. FIG. 3 shows an example. The Z dimension is longitudinal to the patient or a center of rotation of the SPECT detector and/or CT gantry. The topograms may be taken with the view direction (normal vector) substantially along the X direction (lateral topogram 320 with an example 322 of a heart phantom topogram) and the Y direction (top topogram 310 with an example 312 of the heart phantom), perpendicular to each other. The topogram along the X direction projects to a plane in the Y and Z directions. The topogram along the Y direction projects to a plane in the X and Z direction. Both normal vectors are substantially perpendicular to the axis of rotation of the SPECT system (i.e., the Z axis). Substantially is used to account for tolerance in sensing and positioning mechanism, such as +/−2%. Other angles of the topogram view directions relative to the patient and/or each other may be used. For example, a non-perpendicular angle is used. As another example, other directions than along the cardinal (X or Y) axis are used. The normal vectors for the topograms are non-co-linear. The two or more perpendicular or non-co-linear topograms provide more in-depth anatomical information over all three axes (sagittal, coronal, and transverse planes) of the patient as opposed to only one topogram (i.e., the transverse and sagittal planes).

The one, two, or more (few (i.e., less than 5)) topograms are acquired without a full CT acquisition. Fewer topograms than used for a full CT acquisition are acquired, such as two topograms instead of the tens or hundreds for the full CT. The fields of view for the topograms may be different, such as representing the torso of the patient along the Z axis rather than lateral slices of the patient. The time of acquisition is less, such as a few seconds (e.g., fewer than three seconds) or a minute to acquire one topogram, move the gantry, and acquire another topogram. The time is less than the ten minutes or more for a full CT. Rather than a helical or spiral CT scan, a few topograms are acquired from different angles to the patient. The dose of a few topograms may be 1 or less mSv as compared to 6 or more mSv of a full CT scan.

Rather than achieve or approximate CT image quality, the x-ray data for localization and/or generation of the attenuation map is acquired optimized for the localization need. This optimization allows for operation below the sampling for CT image quality. Thus, dose exposure of the patient is less, saving radiation dose.

In act 210, a processor determines an anatomy location of interest from the topograms. The processor may determine the location automatically, such as using landmark detection and/or segmentation. Alternatively, the processor determines the anatomy location based on input from the user, such as a radiologist or imaging technician selecting a landmark in one or more topograms.

The anatomy location is determined in three dimensions. The anatomy location, such as a point or region, is delineated along all three dimensions (X, Y, Z). For example, a center of the heart or other cardiac anatomy (e.g., chamber, valve, or muscle) is determined in three dimensions. The location for subsequent SPECT imaging is located. This localization pinpoints the anatomy of interest as an anatomical or spatial reference for emission tomography imaging.

The determination is from the topograms. In one implementation, the anatomy location is determined from triangulation. The position of the CT relative to the SPECT is known, such as by calibration. The topograms are a two-dimensional projection across the radiation field of the x-ray tube, creating a two-dimensional radiograph. The radiation of the x-ray tube is a simplified fan in transverse and thin in the axial dimension. By detecting or selecting the same location in each of two or more topograms, triangulation is used to determine the location in three dimensions (3D). The known relationship of the CT or x-ray imager to the SPECT system is used to determine the anatomy location in the SPECT system coordinates.

FIG. 3 shows topograms 310, 320 from different directions relative to the patient of the three-dimensional object 330, with an example image 332 as a transverse slice of the three-dimensional object 330. Images 312 and 322 are example images of topograms 310, 320 of a phantom. The representation may be less than CT quality but is sufficient to represent anatomy at a resolution adequate at or similar to the emission tomography imager. The location of the anatomy of interest is determined using triangulation from the topograms 310, 320, such as using landmark detection and/or segmentation of in each topogram and relating those positions from different viewing directions.

The topogram may be distortion corrected to achieve congruency (e.g., length and angle preserving). A point in the topogram (i.e., in the topogram coordinate system) is transferred or translated from the CT gantry coordinate system to the SPECT coordinate system and to the SPECT detector coordinate system. The position of the detectors at different positions or a detector rotated to different positions for the topogram have a separating angle theta, having 2 normal vectors whereby cos(theta)=vec(n1)*vec(n2) intersecting at specific point in the patient coordinate system, which is related to the SPECT coordinate system assuming no movement has occurred between topogram moves and positioning for SPECT scan. Geometric transformation (e.g., matrix multiplication) triangulates the position given that the CT and SPECT fields of view are calibrated by a measurement.

The goal is to estimate a specific location (e.g., a point or region) in patient space. Such a need arises when imaging the heart or other anatomy in particular, such as with a collimator limiting the field of view (e.g., non-parallel hole collimator). Typically, the heart can be located in the projection image with the focusing collimator using noncollinear views, allowing the center for an organ centric rotation of orbit with constant radius about that specified center based on emission data to be located. However, better localization may be provided using the topograms. Anatomy may not be detectable from the nuclear imaging data so localization is provided using the topograms. Using the triangulation approach, with the system design of known emission tomography-to-CT field of view registration, and the collinearity of the topograms, an accurate and precise localization of the anatomy location (e.g., point) in 3D patient emission space is determined by localization in two or more non-co-linear x-ray topograms.

In act 212, the processor controls the emission scanner (SPECT scanner) to focus on the anatomy location. The detector is focused on the anatomy location, such as moving the patient and/or detector to detect emissions from the heart region of the patient.

In one implementation, the collimator and detector are positioned to detect emissions from the anatomy location. This focus of the collimator and detector provides for functional imaging of the anatomy of interest. Where the collimator is a focusing collimator (e.g., non-parallel hole collimator), the focus or focal region of the collimator (e.g., where lines of response defined by the non-parallel holes converge or are denser) is positioned at the anatomy location. The focus or focal region is placed at or to cover the anatomy in three dimensions, such as laterally positioning and positioning in depth. For example, the determined heart center or another heart location is used for a SPECT scan with a focusing collimator.

The determined anatomy location may be used as a landmark for landmark-based registration for, e.g., external CT (CT not part of the SPECT/CT system using a common patient bed) or MR or 3D rigid or non-rigid registration. Mutual information based multi-modal registration performs well when the modalities are within similar anatomical classes (e.g., CT-to-CT, then CT-to-MR, then CT-to-Emission Tomography), so the SPECT system CT-based determined landmark is used to register with an external CT, which is registered to MR.

In act 220, the processor generates an attenuation map. The attenuation map represents attenuation or tissue density per voxel over a three-dimensional region. Since the tissue attenuates the emissions, accounting for the attenuation along each line of response in emission tomography results in more accurate tomography or measurements.

In one implementation, the processor generates the attenuation map from a lowest possible radiation dose from x-rays. The few topograms may be used with a point-by-point concept. The topograms are used to build a delineation of the patient. The patient is, in general, an ellipsoid with two free parameters (two radii). The two topograms are used to measure the radii. Using more topograms may allow for more complex shapes. Alternatively, or additionally, a model-based curve fitting is used, such as using slice-by-slice of the topogram whereby the topogram slice creates N azimuthal data points that can be used for curve fitting using a specific body outline model. This model is fit to the topograms. Radial steps or annuli between rad_min and rad_max may be used (e.g., just the convex hull of the patient is determined with two or more non-collinear topograms). The attenuation by location is provided by the fit model, such as a physics model using water attenuation for bones and air attenuation for the lungs at the energy used for the radioisotope. The topograms show the locations of the lungs. For example, a physics model is used to estimate attenuation for a set of materials, and then the delineated body is filled with a mixture of these materials based on the fitting of the shape model.

The attenuation map is generated from the topograms, such as the one, two, or few topograms used for localization or the 3D patient representation constructed from the topograms. With a few (e.g., 2 or 3) topograms, a proxy three-dimensional attenuation map is generated or constructed. From the delineation on the topograms, a μ-map is constructed or estimated. The topograms from two views are used to construct a 3d approximation of attenuation.

The attenuation coefficient per voxel of the 3D representation is derived from the model fit to the topograms. For example, the 3D x-ray-based anatomy information form the fit model is used to create a μ-map for attenuation correction. The measurements for the CT data are Hounsfield (HU) measurements. For x-ray CT, the transmission spectrum is an attenuated Bremsstrahlung spectrum and thus continuous and depends upon the particular CT scanner and the attenuating body. The HU measurements are used to represent anatomy. Alternatively, the CT data is converted to linear attenuation coefficients. A μ-map is generated from the CT information. The conversion adapts the structural or support information to the CT scanner, the patient, and/or the protocol (see U.S. Pat. No. 6,950,494). Any conversion to a μ-map may be used but is adaptive for best results. This μ-map is used for attenuation correction in emission tomography. Alternatively, the CT data is used without further derivation.

In another approach, the processor generates the attenuation map (e.g., μ-map) using a machine-learned model. An image-to-image model, generative model, encoder-decoder, U-net or other machine learned model is trained to receive an input, such as the topograms, and output the 3D attenuation map. Other inputs may be included, such as emission data, optical (RGB or RGB-depth) images of the patient, and/or patient clinical data (e.g., weight and sex at birth). Various types of machine learning models may be used, such as a neural network (e.g., fully connected neural network, convolutional neural network, and/or DenseNet).

In other approaches, the processor selects, guides, or adapts the machine-learned model used for emission tomography. For example, the topograms are used to select the machine-learned model used in reconstruction for emission tomography. As another example, the topograms are input with the emission data to the machine-learned model, which outputs the emission representation or image. The machine-learned model incorporates or is guided by the topograms to correct for attenuation so that the output representation or image is attenuation corrected. This adapts the machine-learned emission tomography model to the topograms.

The generated attenuation map may be used for localization. For example, a μ-map may have more easily detected landmarks and/or provide for better or more accurate segmentation. This characteristic may also allow for the attenuation map to be used for rigid 3D registration, such as with an external modality (e.g., external to the integrated (shared housing) CT-SPECT systems). A pre-operative or pre-emission scan CT or MR scan may be registered with the patient using the attenuation map from the topograms.

The generated attenuation map may be used for attenuation correction for emission tomography. The attenuation of emissions along lines of response is determined from the attenuation map. The counts or detected emissions are corrected for the amount of attenuation as part of reconstruction in SPECT, PET, or other emission tomography.

In act 230, the emission tomography system (e.g., SPECT system) images the patient. Where localized, the imaging is of the anatomy of interest. For example, the imaging uses the collimator as focused on the heart to detect radiotracer (e.g., PYP) emissions from the heart. The emissions are along lines of response defined by the collimator. Using this spatial information and counts of the emissions at different pixels of the detector at different angles relative to the patient, the distribution of points of emissions in 3D are reconstructed. Since the radiotracer binds to or is up-taken from metabolic operation or function of the patient, the function is reconstructed from the detected emissions.

In act 232, the emission tomography system obtains emissions data. SPECT or PET emission detection is performed on a patient. In alternative embodiments, other functional imaging is performed, such as fMRI or fCT. The emissions data is measurements or counts of emissions from a patient.

The emission data is obtained from detection, from data transfer, or from memory. An emission tomography system provides the emission data directly by detecting from a patient or indirectly by transfer or loading.

The activity concentration in a patient having received a radiotracer or radiotracers is determined as part of reconstruction by the emission tomography system. After ingesting or injecting the radiotracer or tracers into the patient, the patient is positioned relative to an emissions detector, and/or the detector is positioned relative to the patient. Emissions from the radiotracer or tracers within the patient are detected over time. The collimator in front of the detector limits the direction of photons detected by the detector, so each detected emission is associated with an energy and line or cone of possible locations from which the emission occurred (i.e., lines of response).

Raw emission data or preprocessed data is provided for reconstruction. For functional imaging of the patient, the detected or measured emissions from the patient (i.e., from the object) are reconstructed into object space. Any reconstruction may be used. In an iterative optimization, a model of the emission tomography system is used to forward project measurements to object space, and residuals are back projected for correcting a data model for the next iteration. A machine-learned model may be used for the reconstruction or part of the reconstruction optimization. The image object may then be displayed using volume rendering or other imaging techniques.

The constructed representation from the topograms may be used for multi-modal reconstruction. In the multi-modal reconstruction, a functional image of an examined object is reconstructed by considering the spatial or spatial-temporal structure of the object when approximating the functional image according to the acquired emission data. The structure of the object allows separating the object into multiple zones. Each organ or type of tissue is assigned to a separate zone. The volume within each of those zones is treated separately and equally in the reconstruction.

The detected emissions may be reconstructed using the attenuation map (e.g., μ-map). The emissions or image object are corrected to account for attenuation along lines of response in the optimization.

An image is generated from the image object output by the reconstruction. The reconstruction outputs an image object or volume representing the patient from a last iteration. The object space is the space in which the result of the image reconstruction is defined, and which corresponds, for example, to the 3D volume (i.e., field-of-view or “FOV”) that is imaged. This final image object is used for generating an image. The image object is a three-dimensional representation of the detected emissions or activity distribution of the patient. Due to any attenuation correction, the counts or activity concentration for each voxel or location is more accurate. The resulting image is used for diagnosis or prognosis, which is improved by the increased accuracy of the emission tomography system.

The image object is rendered or otherwise used to generate an image. For example, a multi-planar reconstruction or single slice image of a plane is generated. The intersection of one or more planes with the image object is visualized. As another example, a surface or projection rendering is performed for three-dimensional imaging. Other imaging may be used.

The image of the functional information from the reconstruction is displayed alone. Alternatively, an anatomical image is displayed with the functional image. For example, the functional image is overlaid on or displayed with one or more of the topograms. Other combinations may be used, such as displaying CT or MR information from an external scan.

For quantitative SPECT, the image may be an alphanumeric text of a specific uptake value for a location. A graph, chart, or other representation of uptake at multiple locations may be output. The spatial image representing distribution of uptake may use color or brightness modulation to represent a level of uptake by location.

FIG. 4 shows an example benefit of localization based on two topograms. While the low dose is not shown, the accuracy of localization as compared to use of a patient imaging monitor image (see FIG. 1) is shown. The heart map image 400 is based on an experienced imaging technician selecting a region for focused SPECT imaging. The hot regions (light shades) are shifted from center and small relative to the heart map. The heart map image 410 is based on localization from the low dose topograms. The heart is centered on the heart map and encompasses the entire heart map. Better focusing and accurate placement of the emission detection region (focusing) is provided. Adequate heart positioning is provided using the topogram-based localization.

Attenuation correction maps are typically created by acquiring helical or spiral CT imaging data set of the patient or by applying an artificial intelligence (AI or machine learning) algorithm or subsequent mathematical model to the acquired emission data set for prediction of the attenuation map. A full helical or spiral CT image requires giving the patient a higher level of ionizing radiation over the course of a longer scanning duration than the one, two, or few topograms. Using two low dose short duration topograms substantially reduces the level of ionizing radiation exposure to the patient. Additionally, the necessity for a higher ionizing radiation dose helical or spiral CT acquisition may be eliminated for cardiac SPECT or other emission tomography imaging.

In the use of AI algorithms, an ‘averaged learned’ attenuation map is not conducive to patient specific analysis. Such process has the potential to inaccurately calculate attenuation coefficients for different size patients. Attenuation maps are also dependent upon CT scanner protocol parameters, thus, also potentially introducing confounding factors in the calculations where AI is used to predict from emission data. It also remains unclear how well the algorithm can differentiate between systems, scans, or other factors such as the impact of beam hardening effects. Attenuation from topograms is patient specific and accurate.

Studies have attempted to incorporate a lower dose CT protocol technique by reducing the tube voltage, current, adding CT filtering, or applying a sinogram smoothing algorithm. The reduction of the CT protocol techniques requires longer scanning times, is based on the transverse axis, and introduces noise and artifacts. Using topograms applies a 3D mathematically patient-derived attenuation coefficient mapping based on image processing instead while using even lower dose topograms acquired over a short duration.

FIG. 5 shows one embodiment of a system 500 for localizing and/or attenuation mapping for emission imaging. The system 500 uses topograms as a low dose alternative to a full CT scan or inaccuracy of using detected emissions. The method of FIGS. 2 and/or 3 or another method is implemented by the system 500.

The system 500 includes an image processor 560, an emission imaging system 520, x-ray system 540, a memory 590, and a display 580. Additional, different, or fewer components may be provided. In one embodiment, the image processor 560, memory 590, and/or display 580 are part of one of the emission imaging system 520 or the x-ray system 540. In alternative embodiments, the image processor 560, memory 590, and/or display 580 are provided as a workstation, server, or computer separate from the imaging systems 520, 540. The memory 590 is part of a computer or workstation with the image processor 560 or is a remote database, such as a picture archiving and communications system (PACS).

The x-ray scanner or system 540 is a CT, angiography, fluoroscopy, x-ray, or other diagnostic medical x-ray scanner. The x-ray system 540 scans a patient with x-rays to image the anatomy of the interior of the patient. A source transmits energy to the patient. A detector receives signals responsive to the transmitted energy. Any now known or later developed x-ray system 540 may be used. While “imaging” or “image” is used herein, the x-ray system 540 may be used to acquire anatomy data representing the patient without generating or displaying an image on a display device. The x-ray system is configured to acquire one or more topograms (e.g., two topograms).

In one embodiment, the x-ray system 540 is a CT system integrated with or sharing a housing and/or patient bed with the emission imaging system 520. An x-ray source and detector are mounted on a moveable gantry. The x-ray source generates x-rays, some of which pass through the patient. The detector detects the transmitted x-rays that pass through the patient. The energy used, timing, scan angles, and/or other aspects of the CT scan are set for a patient and used to scan the patient. The CT system scans the volume of the patient to capture one, two, or a few topograms. For example, rather than a full helical or spiral CT scan, topograms are captured from two different angles relative to the patient, each as a single detector readout from single x-ray generation.

The x-ray-based scanner or x-ray scanner 540 is configured to acquire one or more x-ray topograms from respective one or more angles to a patient separate from any helical or spiral computed tomography scan. Just the limited number (e.g., two) of topograms are captured for a given imaging session or emission tomography imaging appointment (e.g., within a 4, 8, 12, or 24 hour period).

The emission imaging system or emission scanner 520 is any now known or later developed SPECT scanner, PET scanner, or other scanner configured to detect emissions from a radiotracer in the patient. The emission imaging system 520 includes a detector 522 for detecting emitted radiation passing through a collimator 524 from within the patient. For SPECT, one or more gamma cameras mounted to a moveable gantry are used to detect. A given detector may detect a sequence of events or counts from the same or different locations of the patient.

The emission imaging system 520 is configured to generate a functional image of the heart or other anatomy of the patient. Using localization, the emission imaging system 520 may be focused to scan particular anatomy. For example, a focusing collimator may be positioned to focus on a 3D location or region in the patient for emission imaging. The emission imaging system 520 is configured to generate a functional image of the patient (e.g., of the heart of the patient). The image is localized to the anatomy of interest. The image may be a quantification image. The image may be improved by reconstruction using an attenuation map.

The image processor 560 is a general processor, central processing unit, control processor, graphics processor, digital signal processor, application specific integrated circuit, field programmable gate array, digital circuit, analog circuit, combinations thereof, or other now known or later developed device for reconstructing a patient volume (i.e., object of the patient) from detected emissions. The image processor 560 is a single device or multiple devices operating in serial, parallel, or separately. The image processor 560 is specifically designed or provided for reconstruction but may be a main or general processor of a computer, such as a laptop or desktop computer, or may be a processor for handling tasks in a larger system. The image processor 560 may perform localization, attenuation map generation, reconstruction, and/or control of the imaging systems 520, 540. The image processor 560 may perform other functions.

The image processor 560 is configurable. The image processor 560 is configured by software, firmware and/or hardware. Different software, firmware, and/or instructions are loaded or stored in memory 590 for configuring the image processor 560.

The image processor 560 is configured to localize anatomy relative to the emission scanner and/or generate an attenuation map from the one or more topograms. The image processor 560 is configured to cause the emission scanner or system 520 to detect the emissions based on the localization and/or reconstruct based on the attenuation map.

The image processor 560 is configured to localize and/or generate the attenuation map from a limited number (e.g., just one, two, or a few) of topograms from the X-ray scanner 520. For example, two topograms are used to localize particular anatomy (e.g., the heart) of the patient by triangulation of the anatomy of the patient. The point or region is identified in non-co-linear topograms, providing a point or region in 3D. User input or automated detection are used to identify the point or region. The anatomy may be located using construction (e.g., fit model) from the topograms or model fitting to the topograms in other approaches. This 3D point or region is used to localize the emission imaging. The collimator 524 and/or detector 522 is focused by positioning based on the triangulated anatomy.

As another example, two or a few (e.g., 5 or less) topograms are used to generate an attenuation map. The image processor 560 constructs a 3D representation of the patient from the topograms. The 3D representation (HU units) or a μ-map derived therefrom are used as the attenuation map. A machine-learned model may be used to derive the attenuation map from the topogram with or without other inputs (e.g., emissions) in other approaches. In one approach, a shape model is fit to the patient as represented in the topograms, such as fitting to a boundary or delineation extrapolated to 3D from the topograms. Attenuation values are assigned to the fit model. The image processor 560 then reconstructs the emission data using attenuation correction based on the attenuation map.

The image processor 560 is configured to reconstruct an object representing the patient from the detected emissions. The reconstruction may include attenuation correction based on the attenuation map. Alternatively, attenuation correction is not used. Where localization is used, the reconstructed object is focused on the anatomy of interest, such as due to detection of emissions from the localized region and not from other regions.

The display 580 is a monitor, LCD, plasma, touch screen, printer, or other device for displaying an image for viewing by a user. The display 580 shows one or more images representing function (i.e., representing the activity of the reconstructed object), such as uptake or activity concentration. The image is a quantitative or qualitative SPECT or PET image. The image may be a volume rendering, a multi-planar reconstruction, a cross-section, and/or another image from a final image object. The image represents a distribution of the radionuclide in the patient based on detected emissions from the emission system 520.

The memory 590 is a random-access memory, graphics processing memory, video random access memory, system memory, cache memory, hard drive, optical media, magnetic media, flash drive, buffer, database, combinations thereof, or other now known or later developed memory device for storing data. The memory 590 stores detected emissions (e.g., PET or SPECT detected event data), signals from anatomy scanning (e.g., topograms), localization information (e.g., 3D point), attenuation map, constructed x-ray representation of the patient (e.g., fit model), reconstructed activity, and/or reconstruction information. The memory 590 stores data as processed.

The memory 590 or other memory is a non-transitory computer readable storage medium storing data representing instructions executable by the programmed image processor 560 for reconstruction. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The following is a list of non-limiting illustrative embodiments disclosed herein. The illustrative embodiments below summarize aspects or features of the first, second, and third aspects in the summary above. Aspects or features used for one type of illustrative embodiment (e.g., method or system) may be used in other types.

Illustrative embodiment 1. A method for localization for cardiac imaging in a single photon emission computed tomography (SPECT) system, the method comprising: obtaining first and second topograms of a patient, the first and second topograms comprising x-ray images captured from different angles relative to a patient, the first and second topograms acquired without a full computed tomography acquisition; determining a cardiac location of interest in three dimensions from the first and second topograms; focusing a collimator of the SPECT system based on the cardiac location determined from the first and second topograms; and imaging, using the collimator, the cardiac location by the SPECT system.

Illustrative embodiment 2. The method of illustrative embodiment 1, wherein obtaining comprises obtaining the first and second topograms as the only information from x-rays for the imaging.

Illustrative embodiment 3. The method of any of illustrative embodiments 1-2, wherein obtaining comprises obtaining the first and second topograms with first and second normal vectors, the first normal vector for the first topogram being substantially perpendicular to the second normal vector for the second topogram, both the first and second normal vectors being substantially perpendicular to an axis of rotation of the SPECT system.

Illustrative embodiment 4. The method of any of illustrative embodiments 1-3, wherein obtaining comprises obtaining with a computed tomography imager, and wherein determining comprises determining the cardiac location by triangulation where a known relationship of the computed tomography imager to the SPECT system.

Illustrative embodiment 5. The method of any of illustrative embodiments 1-4, wherein determining comprises constructing a three-dimensional representation of the patient from the first and second topograms and detecting the cardiac location in the three-dimensional representation.

Illustrative embodiment 6. The method of any of illustrative embodiments 1-5, wherein the collimator comprises a non-parallel hole collimator with a focal location, and wherein focusing comprises positioning the focal location at the cardiac location.

Illustrative embodiment 7. The method of any of illustrative embodiments 1-6, wherein imaging comprises detecting emissions by the SPECT system and reconstructing function in the patient from the detected emissions.

Illustrative embodiment 8. The method of any of illustrative embodiments 1-7, further comprising generating an attenuation map from the first and second topograms, and wherein imaging comprises imaging based on the attenuation map.

Illustrative embodiment 9. The method of illustrative embodiment 8, wherein generating comprises delineating a patient shape from the first and second topograms, and deriving attenuation coefficients per voxel using the patient shape.

Illustrative embodiment 10. The method of illustrative embodiment 8, wherein generating comprises generating by a machine-learned model in response to input of the first and second topograms to the machine-learned model.

Illustrative embodiment 11. A system for localization and/or attenuation mapping for emission imaging, the system comprising: an x-ray-based scanner configured to acquire one or more x-ray topograms from respective one or more angles to a patient separate from any helical or spiral computed tomography scan; an emission scanner configured to detect emissions from the patient; and a processor configured to localize anatomy relative to the emission scanner and/or generate an attenuation map from the one or more topograms and cause the emission scanner to detect the emissions based on the localization and/or attenuation map.

Illustrative embodiment 12. The system of illustrative embodiment 11 wherein the x-ray-based scanner comprises a computed tomography scanner integrated with the emission scanner, the computed tomography scanner configured to acquire the one or more x-ray topograms as just first and second topograms, wherein the processor is configured to localize and/or generate from just the first and second topograms.

Illustrative embodiment 13. The system of any of illustrative embodiments 11-12 wherein the processor is configured to localize by triangulation of anatomy of the patient from the one or more topograms, and wherein a collimator of the emission scanner is focused based on the triangulated anatomy.

Illustrative embodiment 14. The system of any of illustrative embodiments 11-13 wherein the processor is configured to generate the attenuation map from a construction of a three-dimensional representation of the patient from the one or more topograms.

Illustrative embodiment 15. The system of any of illustrative embodiments 11-14 wherein the emission scanner is configured to generate a functional image of a heart of the patient where the heart comprises the localized anatomy, the functional image comprising a quantification based on the attenuation map.

Illustrative embodiment 16. A method for attenuation mapping in a single photon emission computed tomography (SPECT) scanner, the method comprising: obtaining first and second topograms of a patient, the first and second topograms comprising x-ray images captured from different angles relative to a patient over a scan of less than three seconds; generating an attenuation map from the first and second topograms, and imaging, by the SPECT scanner, based on the attenuation map.

Illustrative embodiment 17. The method of illustrative embodiment 16 wherein obtaining comprises obtaining comprises obtaining the first and second topograms as the only information from x-rays for the imaging.

Illustrative embodiment 18. The method of any of illustrative embodiments 16-17, wherein obtaining comprises obtaining the first and second topograms with first and second normal vectors, the first normal vector for the first topogram being substantially perpendicular to the second normal vector for the second topogram, both the first and second normal vectors being substantially perpendicular to an axis of rotation of the SPECT scanner.

Illustrative embodiment 19. The method of any of illustrative embodiments 16-18, wherein generating comprises delineating a patient shape from the first and second topograms, and deriving attenuation coefficients per voxel using the patient shape.

Illustrative embodiment 20. The method of any of illustrative embodiments 16-19, further comprising: determining a cardiac location of interest in three dimensions from the first and second topograms; and focusing a collimator of the SPECT scanner based on the cardiac location determined from the first and second topograms; wherein imaging comprises imaging by the SPECT scanner with the collimator as focused.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (WE) CLAIM:

1. A method for localization for cardiac imaging in a single photon emission computed tomography (SPECT) system, the method comprising:
obtaining first and second topograms of a patient, the first and second topograms comprising x-ray images captured from different angles relative to a patient, the first and second topograms acquired without a full computed tomography acquisition;
determining a cardiac location of interest in three dimensions from the first and second topograms;
focusing a collimator of the SPECT system based on the cardiac location determined from the first and second topograms; and
imaging, using the collimator as focused, the cardiac location by the SPECT system.

2. The method of claim 1, wherein obtaining comprises obtaining the first and second topograms as the only information from x-rays for the imaging.

3. The method of claim 1, wherein obtaining comprises obtaining the first and second topograms with first and second normal vectors, the first normal vector for the first topogram being substantially perpendicular to the second normal vector for the second topogram, both the first and second normal vectors being substantially perpendicular to an axis of rotation of the SPECT system.

4. The method of claim 1, wherein obtaining comprises obtaining with a computed tomography imager, and wherein determining comprises determining the cardiac location by triangulation where a known relationship of the computed tomography imager to the SPECT system.

5. The method of claim 1, wherein determining comprises constructing a three-dimensional representation of the patient from the first and second topograms and detecting the cardiac location in the three-dimensional representation.

6. The method of claim 1, wherein the collimator comprises a non-parallel hole collimator with a focal location, and wherein focusing comprises positioning the focal location at the cardiac location.

7. The method of claim 1, wherein imaging comprises detecting emissions by the SPECT system and reconstructing function in the patient from the detected emissions.

8. The method of claim 1, further comprising generating an attenuation map from the first and second topograms, and wherein imaging comprises imaging based on the attenuation map.

9. The method of claim 8, wherein generating comprises delineating a patient shape from the first and second topograms, and deriving attenuation coefficients per voxel using the patient shape.

10. The method of claim 8, wherein generating comprises generating by a machine-learned model in response to input of the first and second topograms to the machine-learned model.

11. A system for localization and/or attenuation mapping for emission imaging, the system comprising:
an x-ray-based scanner configured to acquire one or more x-ray topograms from respective one or more angles to a patient separate from any helical or spiral computed tomography scan;
an emission scanner configured to detect emissions from the patient; and
a processor configured to localize anatomy relative to the emission scanner and/or generate an attenuation map from the one or more topograms and cause the emission scanner to detect the emissions based on the localization and/or attenuation map.

12. The system of claim 11 wherein the x-ray-based scanner comprises a computed tomography scanner integrated with the emission scanner, the computed tomography scanner configured to acquire the one or more x-ray topograms as just first and second topograms, wherein the processor is configured to localize and/or generate from just the first and second topograms.

13. The system of claim 11 wherein the processor is configured to localize by triangulation of anatomy of the patient from the one or more topograms, and wherein a collimator of the emission scanner is focused based on the triangulated anatomy.

14. The system of claim 11 wherein the processor is configured to generate the attenuation map from a construction of a three-dimensional representation of the patient from the one or more topograms.

15. The system of claim 11 wherein the emission scanner is configured to generate a functional image of a heart of the patient where the heart comprises the localized anatomy, the functional image comprising a quantification based on the attenuation map.

16. A method for attenuation mapping in a single photon emission computed tomography (SPECT) scanner, the method comprising:
obtaining first and second topograms of a patient, the first and second topograms comprising x-ray images captured from different angles relative to a patient over a scan of less than three seconds;
generating an attenuation map from the first and second topograms, and
imaging, by the SPECT scanner, based on the attenuation map.

17. The method of claim 16 wherein obtaining comprises obtaining comprises obtaining the first and second topograms as the only information from x-rays for the imaging.

18. The method of claim 16, wherein obtaining comprises obtaining the first and second topograms with first and second normal vectors, the first normal vector for the first topogram being substantially perpendicular to the second normal vector for the second topogram, both the first and second normal vectors being substantially perpendicular to an axis of rotation of the SPECT scanner.

19. The method of claim 16, wherein generating comprises delineating a patient shape from the first and second topograms, and deriving attenuation coefficients per voxel using the patient shape.

20. The method of claim 16, further comprising:
determining a cardiac location of interest in three dimensions from the first and second topograms; and
focusing a collimator of the SPECT scanner based on the cardiac location determined from the first and second topograms;
wherein imaging comprises imaging by the SPECT scanner with the collimator as focused.
